(19) United States
(12) Patent Application Publication
(10) Pub. No.: US 2004/0186077 A1
(43) Pub. Date: Sep. 23, 2004

(54) Novel heteroaryl phosphonates as cardioprotective agents

(75) Inventors: James Diakur, Winnipeg (CA); Wasimul Haque, Edmonton (CA); Wenlian Zhang, Winnipeg (CA); Junzhi Yao, Winnipeg (CA); Vinh Pham, Winnipeg (CA)

Correspondence Address: MERCHANT & GOULD PC, P.O. BOX 2903, MINNEAPOLIS, MN, 55402-0903, US

(73) Assignee: Medicure International Inc., St. James (BB)

(21) Appl. No.: 10/391,056

(22) Filed: Mar. 17, 2003

(51) Int. Cl. A61K 31/675; A61K 31/44

(52) U.S. Cl. 514/89; 514/277; 546/22

(57) ABSTRACT

The invention teaches compound of general formula:

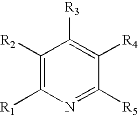

Wherein:

R₁ is selected from H and CH₃, and R₂ is selected from H and OH, or R₁ and R₂ together form an optionally substituted phenyl ring which is fused to the pyridine ring; and

R₃ is selected from H, CH₃, CH₂OH and

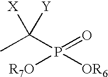

R₄ is selected from H, CH₃, CH₂OH,

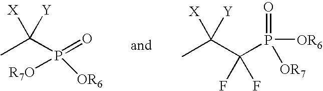

R₅ is selected from H, phenyl, halogen-substituted phenyl and

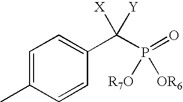

Wherein R₆ and R₇ are each independently selected from H, Na⁺, K⁺, alkyl and optionally substituted aryl, and X and Y are each independently selected from H, OH and F, or at least one of X and Y is an heteroatom and together with R₃ forms a bridge with the proviso that R₄ is

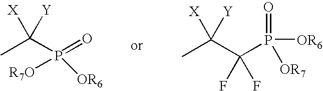

and N-oxides thereof, and biologically acceptable salts thereof, related compounds, related pharmaceutical compositions, and methods for treating various disorders using such compositions.

This invention relates to α-substituted phosphonates, to their preparation, to pharmaceutical compositions thereof, and to treatments for cardiovascular and related diseases, for example, hypertrophy, hypertension, congestive heart failure, myocardial ischemia, arrhythmia, heart failure subsequent to myocardial infarction, myocardial infarction, ischemia reperfusion injury, and diseases that arise from thrombotic and prothrombotic states in which the coagulation cascade is activated; and treatments for diabetes mellitus and related diseases, for example, hyperinsulinemia, diabetes-induced hypertension, obesity, insulin resistance, and damage to blood vessels, eyes, kidneys, nerves, autonomic nervous system, skin connective tissue, or immune system.

Certain phosphonate compounds are known to play a role in mammalian health. (See for example PCT/CA01/00265.)

### SUMMARY OF THE INVENTION

The present invention provides for phosphonate heterocycle analogues. In one aspect, the present invention includes compounds of general formula I below, and N-oxides thereof, and biologically acceptable salts thereof:

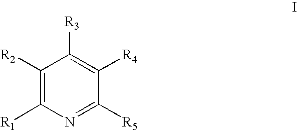

Wherein:

R₁ is selected from H and CH₃, and R₂ is selected from H and OH, or R₁ and R₂ together form an optionally substituted phenyl ring which is fused to the pyridine ring; and

R₃ is selected from H, CH₃, CH₂OH and

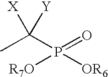

R₄ is selected from H, CH₃, CH₂OH,

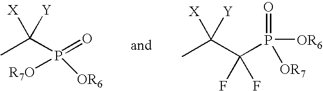

R₅ is selected from H, phenyl, halogen-substituted phenyl and

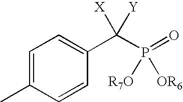

Wherein R₆ and R₇ are each independently selected from H, Na⁺, K⁺, alkyl and optionally substituted aryl, and X and Y are each independently selected from H, OH and F, or at least one of X and Y is an heteroatom and together with R₃ forms a bridge with the proviso that R₄ is

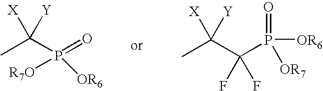

In another aspect, the invention is directed to pharmaceutical compositions that include a pharmaceutically acceptable carrier and a therapeutically effective amount of a compound of formula I or an N-oxide thereof.

In another aspect, the invention is directed to a method of treating cardiovascular and related diseases, for example, hypertension, hypertrophy, arrhythmia, congestive heart failure, myocardial ischemia, heart failure subsequent to myocardial infarction, myocardial ischemia, ischemia reperfusion injury, and diseases that arise from thrombotic and prothrombotic states in which the coagulation cascade is activated by administering a therapeutically effective amount of a compound of formula I and/or an N-oxide thereof in a unit dosage form. For such a method, a compound of formula I and/or an N-oxide thereof can be administered alone or concurrently with a known therapeutic cardiovascular agent, for example, angiotensin converting enzyme inhibitor, an angiotensin II receptor antagonist, a vasodilator, a diuretic, an α-adrenergic receptor antagonist, a β-adrenergic receptor antagonist, an antioxidant, or a mixture thereof.

In still another aspect, the invention is directed to a method of treating diabetes mellitus and related diseases, for example, hyperinsulinemia, insulin resistance, obesity, diabetes-induced hypertension, and damage to eyes, kidneys, blood vessels, nerves, autonomic nervous system, skin, connective tissue, or immune system, by administering a therapeutically effective amount of a compound of formula I and/or an N-oxide thereof in a unit dosage form. For such a method, a compound of formula I and/or an N-oxide thereof can be administered alone or concurrently with known medicaments suitable for treating diabetes mellitus and related diseases, for example, insulin, hypoglycemic drugs, or a mixture thereof.

### BRIEF DESCRIPTION OF THE FIGURES

FIG. 1 is a graphical depiction of the area at risk as determined in Example 39.

FIG. 2 is a graphical depiction of the infarct size as determined in Example 39.

### DETAILED DESCRIPTION OF THE PREFERRED EMBODIMENTS

The present invention provides for a-substituted phosphonates of formula I and/or an N-oxide thereof, including, for example, [Hydroxy-(5-hydroxy4-hydroxymethyl-6-methyl-2-phenyl-pyridin-3-yl)-methyl]-phosphonic acid; {[2-(4-Fluoro-phenyl)-5-hydroxy-4-hydroxymethyl-6-methyl-pyridin-3-yl]-hydroxymethyl}-phosphonic acid; [Hydroxy-(4-pyridin-2-yl-phenyl)-methyl]-phosphonic acid; [Fluoro-(4-pyridin-2-yl-phenyl)-methyl]-phosphonic acid; (Hydroxy-quinolin-3-yl-methyl)-phosphonic acid; (Fluoro-quinolin-3-yl-methyl)phosphonic acid; [Hydroxy-(5-hydroxy-4,6-dimethyl-pyridin-3-yl)-methyl]-phosphonic acid; (Hydroxy-pyridin-4-yl-methyl)-phosphonic acid; (Hydroxy-pyridin-3-yl-methyl)-phosphonic acid; (3,7-Dihydroxy-6-methyl-1,3-dihydro-furo[3,4-c]pyridin-3-yl)phosphonic acid; [(3,7-Dihydroxy-6-methyl-1,3-dihydro-furo[3,4-c]pyridin-3-yl)-difluoromethyl]-phosphonic acid; nicotinyl phosphonates, N-oxides and phosphonate esters. N-oxides of compounds of formula I and/or an N-oxide thereof and biologically acceptable salts of compounds of formula I and N-oxides thereof are also contemplated and fall within the scope of the invention.

Cardiovascular and related diseases include, for example, hypertension, hypertrophy, congestive heart failure, heart failure subsequent to myocardial infarction, arrhythmia, myocardial ischemia, myocardial infarction, ischemia reperfusion injury, and diseases that arise from thrombotic and prothrombotic states in which the coagulation cascade is activated.

Drug therapies, using known active ingredients such as vasodilators, angiotensin II receptor antagonists, angiotensin converting enzyme inhibitors, diuretics, antithrombolytic agents, αor β-adrenergic receptor antagonists, α-adrenergic receptor antagonists, calcium channel blockers, and the like, are available for treating cardiovascular and related diseases.

Diabetes mellitus and related diseases include hyperinsulinemia, insulin resistance, obesity, diabetes-induced hypertension, and damage to blood vessels, eyes, kidneys, nerves, autonomic nervous system, skin, connective tissue, and immune system.

Available treatments include weight control, exercise, diet, and drug therapy. Drug therapy for type I diabetes mellitus requires the administration of insulin; however, drug therapy for type II diabetes mellitus usually involves the administration of insulin and/or oral hypoglycemic drugs to lower blood glucose levels. If the oral hypoglycemic drugs fail to control blood sugar, then insulin, either alone or concurrently with the hypoglycemic drugs, will usually be administered.

The invention is generally directed to a-substituted phosphonates such as, for example, [Hydroxy-(5-hydroxy-4-hydroxymethyl-6-methyl-2-phenyl-pyridin-3-yl)-methyl]-phosphonic acid; {[2-(4-Fluoro-phenyl)-5-hydroxy-4-hydroxymethyl-6-methyl-pyridin-3-yl]-hydroxymethyl}-phosphonic acid; [Hydroxy-(4-pyridin-2-yl-phenyl)-methyl]-phosphonic acid; [Fluoro-(4-pyridin-2-yl-phenyl)-methyl]-phosphonic acid; (Hydroxy-quinolin-3-yl-methyl)-phosphonic acid; (Fluoro-quinolin-3-yl-methyl)-phosphonic acid; [Hydroxy-(5-hydroxy-4,6-dimethyl-pyridin-3-yl)-methyl]-phosphonic acid; (Hydroxy-pyridin-4-yl-methyl)-phosphonic acid; (Hydroxy-pyridin-3-yl-methyl)-phosphonic acid; (3,7-Dihydroxy-6-methyl-1,3-dihydro-furo[3,4-c]pyridin-3-yl)-phosphonic acid; [(3,7-Dihydroxy-6-methyl-1,3-dihydro-furo[3,4-c]pyridin-3-yl)-difluoromethyl]-phosphonic acid; nicotinyl phosphonates, N-oxides and phosphonate esters, compositions including these analogues, and methods of administering pharmaceutical compositions containing a therapeutically effective amount of at least one of these analogues to treat cardiovascular and related diseases or diabetes and related diseases.

To enhance absorption from the digestive tract and across biological membranes, polar groups on a drug molecule can be blocked with lipophilic functions that will be enzymatically cleaved off from the drug after absorption into the circulatory system. Lipophilic moieties can also improve site-specificity and biovailability of the drug. The speed at which the blocking groups are removed can be used to control the rate at which the drug is released. The blocking of polar groups on the drug can also slow first-pass metabolism and excretion. An ester may be employed as a blocking group that is readily hydrolyzed from the drug by endogenous esterases.

In one aspect, the present invention provides compounds that are α-substituted phosphonates. Such compounds are represented by the general formula I:

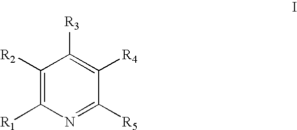

Wherein:

R₁ is selected from H and CH₃, and R₂ is selected from H and OH, or R₁ and R₂ together form an optionally substituted phenyl ring which is fused to the pyridine ring; and

R₃ is selected from H, CH₃, CH₂OH and

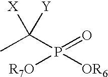

R₄ is selected from H, CH₃, CH₂OH,

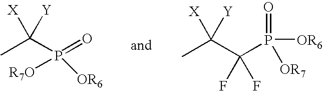

R₅ is selected from H, phenyl, halogen-substituted-phenyl and

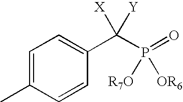

Wherein R₆ and R₇ are each independently selected from H, Na⁺, K⁺, alkyl and optionally substituted aryl, and X and Y are each independently selected from H, OH and F, or at least one of X and Y is an heteroatom and together with R₃ forms a bridge with the proviso that R₄ is

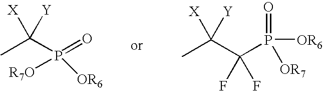

In preferred embodiments, the halogen-substituted phenyl of the compounds according to the invention is a fluoro-substituted phenyl and more preferrably a para-fluoro-substituted phenyl.

The hetero atom of the compounds according to the invention is preferrably oxygen. Optionally, the heteroatom can be sulfur.

According to preferred embodiments, the bridge in the compounds is a C₁ to C₃ bridge, preferrably a methylene bridge. The alkyl group can be a C₁ to C₆ straight or branched alkyl group, preferrably, the alkyl group is t-butyl. In other preferred embodiments, the aryl group can be phenyl or naphthyl.

Without limiting the generality of the invention, embodiments of interest include:

Embodiment 1: Compounds of general formula I, wherein R₁, R₂, R_{4,} R₅ are all H and R₃ is

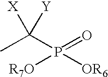

and corresponding N-oxides.

Embodiment 2: compounds of general formula I, wherein R₁, R₂, R₃, R₅ are all H and R₄ is

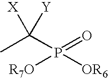

and corresponding N-oxides.

Embodiment 3: compound of general formula I, wherein R₁, R₂, R₃, R₄ are all H and R₅ is

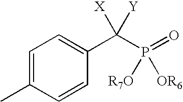

and corresponding N-oxides.

Embodiment 4: compounds of general formula I, wherein R₁ and R₂ together form an optionally substituted phenyl ring which is fused to the pyridine ring; R₃ and R₅ are both H; and R₄ is

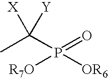

and corresponding N-oxides.

Embodiment 5: compounds of general formula I, wherein R₁, and R₃ are both CH₃; R₂ is OH; R₅ is H; and R₄ is

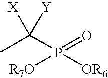

and corresponding N-oxides.

Embodiment 6: compounds of general formula I, wherein R₁ and R₄ are both CH₃; R₂ is OH; R₅ is H; and R₃ is

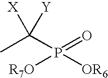

and corresponding N-oxides.

Embodiment 7: compounds of general formula I, wherein R₁ is CH₃; R₂ is OH; R₃ is CH₂OH; R₅ is H; and R₄

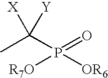

is and corresponding N-oxides.

Embodiment 8: compounds of general formula I, wherein R₁ is CH₃; R₂ is OH; R₃ is CH₂OH; R₅ is H; and R₄ is

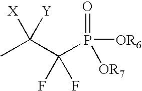

and corresponding N-oxides.

Embodiment 9: compounds of general formula I, wherein R₁ is CH₃; R₂ is OH; R₃ is CH₂OH; R₅ is C₆H₅; and R₄ is

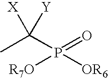

and corresponding N-oxides.

Embodiment 10: compound of general formula I, wherein R₁ is CH₃; R₂ is OH; R₃ is CH₂OH; R₅ is p-C₆H₄F; and R₄ is

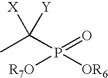

and corresponding N-oxides.

Other examples of compounds of interest include hemiketal forms of these compounds and nicotinic acid derivatives.

Pharmaceutically acceptable acid addition salts of the compounds of formula I and/or an N-oxides thereof include salts derived from inorganic acids such as hydrochloric, nitric, phosphoric, sulfuric, hydrobromic, hydriodic, hydrofluoric, phosphorus, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, tartrate, methanesulfonate, and the like. Also contemplated are salts of amino acids such as arginate and the like and gluconate, galacturonate, N-methyl glutamine, etc. (see, e.g., Berge et al., J. Pharm. Sci., 66:1-19 (1977)).

The acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

One skilled in the art would recognize variations in the sequence of steps and would recognize variations in the appropriate reaction conditions from the analogous reactions shown or otherwise known that can be appropriately used in the described processes to make the compound of formula I and/or an N-oxide thereof herein.

The products of the reactions described herein are isolated by conventional means such as extraction, distillation, chromatography, and the like.

It is to be understood that the recitation of numerical ranges by endpoints includes all numbers and fractions subsumed within that range (e.g. 1 to 5 includes 1, 1.5, 2, 2.75, 3, 3.80, 4, and 5).

It is to be understood that all numbers and fractions thereof are presumed to be modified by the term “about.”

It is to be understood that “a,” “an,” and “the” include plural referents unless the content clearly dictates otherwise. Thus, for example, reference to a composition containing “a compound” includes a mixture of two or more compounds.

It is to be understood that some of the compounds described herein contain one or more asymmetric centers and may thus give rise to enantiomers, diasteriomers, and other stereoisomeric forms which may be defined in terms of absolute stereochemistry as (R)— or (S)—. The present invention is meant to include all such possible diasteriomers and enantiomers as well as their racemic and optically pure forms. Optically active (R)— and (S)— isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise all tautomeric forms are intended to be included. The present invention is meant to include all forms of the hemiketals/keto alcohols disclosed herein.

In accordance with the present invention, the compounds of formula I and/or an N-oxide thereof can be used in the treatment of cardiovascular and related diseases; and in the treatment of diabetes mellitus and related diseases.

Cardiovascular and related diseases include, for example, hypertension, hypertrophy, congestive heart failure, heart failure subsequent to myocardial infarction, arrhythmia, myocardial ischemia, myocardial infarction, ischemia reperfusion injury, and diseases that arise from thrombotic and prothrombotic states in which the coagulation cascade is activated.

Diabetes mellitus and related diseases include, for example, hyperinsulinemia, insulin resistance, obesity, diabetes-induced hypertension, and damage to blood vessels, eyes, kidneys, nerves, autonomic nervous system, skin, connective tissue, and immune system.

“Treatment” and “treating” as used herein include preventing, inhibiting, alleviating, and healing cardiovascular and related diseases; diabetes mellitus and related diseases; or symptoms thereof. Treatment can be carried out by administering a therapeutically effective amount of a compound of the invention. A “therapeutically effective amount” as used herein includes a prophylactic amount, for example, an amount effective for preventing or protecting against cardiovascular and related diseases; diabetes mellitus and related diseases; or symptoms thereof, and amounts effective for alleviating or healing cardiovascular and related diseases; or diabetes mellitus and related diseases; or symptoms thereof.

A physician or veterinarian of ordinary skill readily determines a subject who is exhibiting symptoms of any one or more of the diseases described above. Regardless of the route of administration selected, the compound of formula I and/or an N-oxide thereof or a pharmaceutically acceptable acid addition salt thereof can be formulated into pharmaceutically acceptable unit dosage forms by conventional methods known to the pharmaceutical art. An effective but nontoxic quantity of the compound is employed in treatment. The compounds can be administered in enteral unit dosage forms, such as, for example, tablets, sustained-release tablets, enteric coated tablets, capsules, sustained-release capsules, enteric coated capsules, pills, powders, granules, solutions, and the like. They can also be administered parenterally, such as, for example, subcutaneously, intramuscularly, intradermally, intramammarally, intravenously, and other administrative methods known in the art.

Although it is possible for a compound of the invention to be administered alone in a unit dosage form, preferably the compound is administered in admixture as a pharmaceutical composition. A pharmaceutical composition comprises a pharmaceutically acceptable carrier and a compound of formula I and/or an N-oxide thereof, or a pharmaceutically acceptable acid addition salt thereof. A pharmaceutically acceptable carrier includes, but is not limited to, physiological saline, ringers, phosphate-buffered saline, and other carriers known in the art. Pharmaceutical compositions can also include additives, for example, stabilizers, antioxidants, colorants, excipients, binders, thickeners, dispersing agents, readsorpotion enhancers, buffers, surfactants, preservatives, emulsifiers, isotonizing agents, and diluents. Pharmaceutically acceptable carriers and additives are chosen such that side effects from the pharmaceutical compound are minimized and the performance of the compound is not canceled or inhibited to such an extent that treatment is ineffective.

Methods of preparing pharmaceutical compositions containing a pharmaceutically acceptable carrier and a compound of formula I and/or an N-oxide thereof or a pharmaceutically acceptable acid addition salt thereof are known to those of skill in the art in light of the disclosure herein. All methods can include the step of bringing the compound of the invention in association with the carrier and additives. The formulations generally are prepared by uniformly and intimately bringing the compound of the invention into association with a liquid carrier or a finely divided solid carrier or both, and then, if necessary, shaping the product into the desired unit dosage form.

The ordinarily skilled physician or veterinarian will readily determine and prescribe the therapeutically effective amount of the compound to treat the disease for which treatment is administered. In so proceeding, the physician or veterinarian could employ relatively low dosages at first, subsequently increasing the dose until a maximum response is obtained. Typically, the particular disease, the severity of the disease, the compound to be administered, the route of administration, and the characteristics of the mammal to be treated, for example, age, sex, and weight, are considered in determining the effective amount to administer.

The compound also can be administered to treat cardiovascular and related diseases, for example, hypertrophy, hypertension, congestive heart failure, heart failure subsequent to myocardial infarction, myocardial ischemia, ischemia reperfusion injury, or arrhythmia. Preferably, the cardiovascular disease treated is hypertrophy or congestive heart failure. Still preferably, the cardiovascular disease treated is arrhythmia. Also preferably, the cardiovascular disease treated is ischemia reperfusion injury.

The compound can also be administered to treat cardiovascular diseases and other diseases that arise from thrombotic and prothrombotic states in which the coagulation cascade is activated, such as, for example, deep vein thrombosis, disseminated intravascular coagulopathy, Kasabach-Merritt syndrome, pulmonary embolism, myocardial infarction, stroke, thromboembolic complications of surgery, and peripheral arterial occlusion. A compound of the invention may also be useful in the treatment of adult respiratory distress syndrome, septic shock, septicemia, or inflammatory responses, such as edema and acute or chronic atherosclerosis, because thrombin has been shown to activate a large number of cells outside of the coagulation process, such as, for example, neutrophils, fibroblasts, endothelial cells, and smooth muscle cells.

Moreover, the compound can be administered concurrently with compounds that are already known to be suitable for treating the above-identified diseases. For example, methods of the invention include concurrently administering a compound of formula I and/or an N-oxide thereof, a pharmaceutically acceptable acid addition salt thereof, or a mixture thereof with a therapeutic cardiovascular compound to treat hypertrophy, hypertension, congestive heart failure, heart failure subsequent to myocardial infarction, myocardial ischemia, ischemia reperfusion injury, arrhythmia, or myocardial infarction. Preferably the cardiovascular disease treated is hypertrophy or congestive heart failure. Still preferably, the cardiovascular disease treated is arrhythmia. Also preferably, the cardiovascular disease treated is ischemia reperfusion injury.

Therapeutic cardiovascular compounds that can be concurrently administered with at least one compound of the invention include an angiotensin converting enzyme inhibitor, an angiotensin 11 receptor antagonist, a calcium channel blocker, an antithrombolytic agent, a P-adrenergic receptor antagonist, a vasodilator, a diuretic, an a-adrenergic receptor antagonist, an antioxidant, a statin drug, an angiotension receptor blocker and a mixture thereof. A compound of the invention also can be concurrently administered with PPADS (pyridoxal phosphate-6-azophenyl-2′,4′-disulphonic acid), also a therapeutic cardiovascular compound, or with PPADS and another known therapeutic cardiovascular compound as already described.

Preferably a therapeutic cardiovascular compound, which is concurrently administered with a compound of formula I and/or an N-oxide thereof, a pharmaceutically acceptable acid addition salt thereof, or a mixture thereof, is an angiotensin converting enzyme inhibitor, an angiotensin II receptor antagonist, or a diuretic. Still preferably, the therapeutic cardiovascular compound is an α-adrenergic receptor antagonist. Also preferably, the therapeutic cardiovascular compound is a calcium channel blocker.

These therapeutic cardiovascular compounds are generally used to treat cardiovascular and related diseases as well as symptoms thereof. A skilled physician or veterinarian readily determines a subject who is exhibiting symptoms of any one or more of the diseases described above and makes the determination about which compound is generally suitable for treating specific cardiovascular conditions and symptoms.

For example, myocardial ischemia can be treated by the administration of, for example, angiotensin converting enzyme inhibitor, an angiotensin II receptor antagonist, a calcium channel blocker, an antithrombolytic agent, a β-adrenergic receptor antagonist, a diuretic, an α-adrenergic receptor antagonist, or a mixture thereof. In some instances, congestive heart failure can be treated by the administration of, for example, angiotensin converting enzyme inhibitor, an angiotensin II receptor antagonist, a calcium channel blocker, a vasodilator, a diuretic, or a mixture thereof.

Myocardial infarction can be treated by the administration of, for example, angiotensin converting enzyme inhibitor, a calcium channel blocker, an antithrombolytic agent, a β-adrenergic receptor antagonist, a diuretic, an α-adrenergic receptor antagonist, or a mixture thereof.

Hypertension can be treated by the administration of, for example, angiotensin converting enzyme inhibitor, a calcium channel blocker, a β-adrenergic receptor antagonist, a vasodilator, a diuretic, an α-adrenergic receptor antagonist, or a mixture thereof.

Moreover, arrhythmia can be treated by the administration of, for example, a calcium channel blocker, a β-adrenergic receptor antagonist, or a mixture thereof.

Antithrombolytic agents are used for reducing or removing blood clots from arteries.

Hypertrophy can be treated by the administration of, for example, an angiotensin converting enzyme inhibitor, an angiotensin II receptor antagonist, a calcium channel blocker, or a mixture thereof.

Ischemia reperfusion injury can be treated by the administration of, for example, an angiotensin converting enzyme inhibitor, an angiotensin II receptor antagonist, a calcium channel blocker, or a mixture thereof.

Known angiotensin converting enzyme inhibitors include, for example, captopril, enalapril, lisinopril, benazapril, fosinopril, quinapril, ramipril, spirapril, imidapril, and moexipril.

Examples of known angiotensin II receptor antagonists include both angiotensin I receptor subtype antagonists and angiotensin II receptor subtype antagonists. Suitable antiotensin II receptor antagonists include losartan and valsartan.

Suitable calcium channel blockers include, for example, verapamil, diltiazem, nicardipine, nifedipine, amlodipine, felodipine, nimodipine, and bepridil.

Antithrombolytic agents known in the art include antiplatelet agents, aspirin, and heparin.

Examples of known β-adrenergic receptor antagonists include atenolol, propranolol, timolol, and metoprolol.

Suitable vasodilators include, for example, hydralazine, nitroglycerin, and isosorbide dinitrate.

Suitable diuretics include, for example, furosemide, diuril, amiloride, and hydrodiuril.

Suitable α-adrenergic receptor antagonists include, for example, prazosin, doxazocin, and labetalol.

Suitable antioxidants include vitamin E, vitamin C, and isoflavones.

A compound of formula I and/or an N-oxide thereof, a pharmaceutically acceptable acid addition salt thereof, or a mixture thereof and a therapeutic cardiovascular compound can be administered concurrently. “Concurrent administration” and “concurrently administering” as used herein includes administering a compound of the invention and a therapeutic cardiovascular compound in admixture, such as, for example, in a pharmaceutical composition or in solution, or as separate compounds, such as, for example, separate pharmaceutical compositions or solutions administered consecutively, simultaneously, or at different times but not so distant in time such that the compound of the invention and the therapeutic cardiovascular compound cannot interact and a lower dosage amount of the active ingredient cannot be administered.

A compound of formula I and/or an N-oxide thereof, a pharmaceutically acceptable acid addition salt thereof, or a mixture thereof also can be administered to treat diabetes mellitus and related diseases. Preferably the disease treated is type I diabetes, type II diabetes, or obesity. Also preferably, the disease treated is damage to blood vessels, eyes, kidneys, nerves, autonomic nervous system, skin, connective tissue, or immune system. Still preferably, the disease treated is insulin resistance or hyperinsulinemia. And preferably, the disease treated is diabetes-induced hypertension.

The method of the invention also includes concurrently administering a compound of formula I and/or an N-oxide thereof, a pharmaceutically acceptable acid addition salt thereof, or a mixture thereof with insulin and/or a hypoglycemic compound to treat diabetes mellitus and related diseases. The compound can be administered concurrently with insulin and/or a hypoglycemic compound to treat type I diabetes, type II diabetes, or obesity. Preferably the compound can be administered concurrently with insulin and/or hypoglycemic compound to treat damage to blood vessels, eyes, kidneys, nerves, autonomic nervous system, skin, connective tissue, or immune system. Still preferably, the compound can be administered concurrently with insulin and/or hypoglycemic compound to treat insulin resistance or hyperinsulinemia. Also preferably, the compound can be administered concurrently with insulin and/or hypoglycemic compound to treat diabetes-induced hypertension.

A compound typically can be administered concurrently with insulin to treat type I diabetes, type II diabetes, and related conditions and symptoms. For type II diabetes, insulin resistance, hyperinsulinemia, diabetes-induced hypertension, obesity, or damage to blood vessels, eyes, kidneys, nerves, autonomic nervous system, skin, connective tissue, or immune system, a compound can be administered concurrently with a hypoglycemic compound instead of insulin. Alternatively, a compound can be administered concurrently with insulin and a hypoglycemic compound to treat type II diabetes, insulin resistance, hyperinsulinemia, diabetes-induced hypertension, obesity, or damage to blood vessels, eyes, kidneys, nerves, autonomic nervous system, skin, connective tissue, or immune system.

“Concurrent administration” and “concurrently administering” as used herein includes administering a compound of formula I and/or an N-oxide thereof, a pharmaceutically acceptable acid addition salt thereof, or a mixture thereof and insulin and/or a hypoglycemic compound in admixture, such as, for example, in a pharmaceutical composition, or as separate compounds, such as, for example, separate pharmaceutical compositions administered consecutively, simultaneously, or at different times. Preferably, if the compound and insulin and/or hypoglycemic compound are administered separately, they are not administered so distant in time from each other that the compound and the insulin and/or hypoglycemic compound cannot interact and a lower dosage amount of insulin and/or hypoglycemic compound cannot be administered.

Suitable hypoglycemic compounds include, for example, metformin, acarbose, acetohexamide, glimepiride, tolazamide, glipizide, glyburide, tolbutamide, chlorpropamide, and a mixture thereof. Preferably the hypoglycemic compound is tolbutamide.

This invention will be further characterized by the following examples. These examples are not meant to limit the scope of the invention, which has been fully set forth in the foregoing description. Variations within the scope of the invention will be apparent to those skilled in the art.

### EXAMPLE 1

The structure can be represented by formula 1:

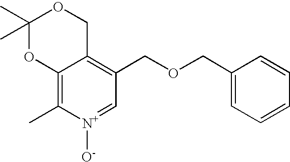

### Synthesis of 5-Benzvioxymethyl-2,2,8-trimethyl-4H-[1,3]dioxino[4,5]pyridine7-oxide (1)

The starting 3,4-isopropylidene 5-O benzyl pyridoxol (2.1 g, 7.01 mmol), (Korytnyk et al., J. Med. Chem., 13 187, (1970)), was dissolved in anhydrous dichloromethane (50 mL), and then 3-chloroperbenzoic acid (3.14 g, 14.02 mmol) was added to the solution. The mixture was stirred at 0° C. for 30 min and then the reaction mixture allowed to warm to room temperature and stirred for a additional 3 h. Evaporation of the solvent followed by purification of the crude product by silica gel column chromatography using ethyl acetate:methanol 4:1 as an eluent gave 2.1 g (95%) of the N-oxide 1.

¹H NMR (CDCl₃) δ 7.94 (s, 1H), 7.3-7.2 (m, 5H), 4.79 (s, 2H), 4.51 (s, 2H), 4.34 (s, 2H), 2.40 (s, 3H), 1.54 (s, 6H).

### EXAMPLE 2

The structure can be represented by formula 2:

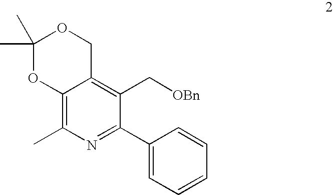

### Synthesis of 5-Benzvioxymethyl-2,2,8-trimethyl-6-phenyl-4H-[1,3]dioxino[4,5-c]pyridine (2)

To a solution of N-oxide 1 (155 mg, 0.49 mmol) in tetrahydrofuran (5 mL), was added isobutylchloroformate (0.07 mL, 0.54 mmol). The reaction mixture was stirred at room temperature under nitrogen for 10 min. After this time, the mixture was cooled to −78° C., and following the addition of phenyl magnesium chloride (0.49 mL, 0.98 mmol), the reaction mixture was stirred for an additional 1 h at this temperature. The reaction was then allowed to warm to room temperature and stirred for an additional 2 h. The reaction mixture was then diluted with diethyl ether (50 mL), and the organic layer washed exhaustively with saturated sodium bicarbonate, dried (MgSO₄) and evaporated to dryness to give crude 2. Purification by silica gel column chromatography using hexane: ethyl acetate 9:1 as eluent gave 36 mg (20%) of pure 2.

¹H NMR (CDCl₃) δ 7.53-7.49 (m, 2H), 7.40-7.29 (m, 8H), 4.96 (s, 2H), 4.45 (s, 2H), 4.36 (s, 2H), 2.47 (s, 3H), 1.59 (s, 6H).

¹³C NMR (CDCl₃) δ 147.37, 145.14, 140.02, 137.62, 129.38, 128.47, 128.12, 128.05, 127.91, 127.65, 127.19, 123.73, 99.58, 72.78, 66.58, 59.13, 24.87, 18.74.

### EXAMPLE 3

The structure can be represented by formula 3:

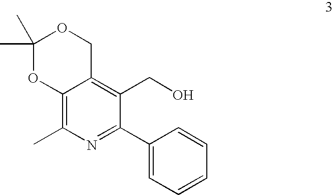

### Synthesis of (2,2,8-Trimethyl-6-phenyl-4H-[1,3]dioxino[4,5-clpyridin-5-yl)-methanol (3)

To a solution of compound 2 (273 mg, 0.63 mmol) in methanol (10 mL), was added 10% Pd/C (550 mg, 50% moisture content) and the mixture was hydrogenated at 40 psi for 72 h. Filtration through Celite® to remove the catalyst followed by purification of the crude product by silica gel column chromatography using hexane:ethyl acetate 9:1 to 1:1 as eluent gave the desired compound 3 in appreciable yields. (112 mg, 62%).

¹H NMR (CDCl₃) δ:7.43-7.37 (m, 5H), 5.02 (s, 2H), 4.47 (s, 2H), 2.44 (s, 3H), 1.59 (s, 6H).

### EXAMPLE 4

The structure can be represented by formula 4:

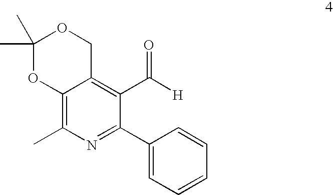

### Synthesis of 2,2,8-Trimethyl-6-phenyl-4H-[1,3]dioxino[4,5-c]pyridine-5-carbaldehyde (4)

The alcohol 3 (112 mg, 0.39 mmol) was dissolved in toluene (10 mL). To the solution was added manganese dioxide (580 mg, 3.92 mmol), and the reaction mixture was stirred at 80° C. overnight. Filtration of the reaction mixture through Celite® gave a clear solution, which was evaporated to dryness. Purification by column chromatography over silica gel using hexane:ethyl acetate 9:1 as eluent afforded pure 4 (67 mg, 60%).

¹H NMR (CDCl₃) δ 9.92 (s, 1H), 7.51-7.45 (m, 5H), 5.21 (s, 2H), 2.55 (s, 3H), 1.60 (s, 6H).

### EXAMPLE 5

The structure can be represented by formula 5:

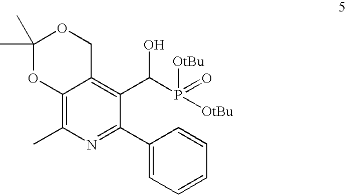

### Synthesis of [Hydroxy-(2,2,8-trimethyl-6-phenyl-4H-[1,3]dioxino[4,5c]pyridin-5-yl)-methyl]-phosphonic acid di-tert-butyl ester (5)

A solution of di-tert-butyl phosphite (587 mg, 2.9 mmol) was slowly added to a suspension of NaH (60% in mineral oil, 93 mg, 2.32 mmol) in THF (10 mL), and the mixture stirred at room temperature for 30 min. To this reaction mixture was added a solution of the aldehyde 4 (329 mg, 1.16 mmol) in THF (10 mL), and the mixture was allowed to stir overnight to complete the reaction. The reaction was then diluted with diethyl ether (100 mL), and washed with aqueous saturated sodium bicarbonate. The ethereal layer was dried over anhydrous magnesium sulfate and evaporated to dryness. Purification of the crude mixture by silica gel column chromatography using hexane:ethyl ether:ammonia 33:66:1 as eluent gave phosphonate 5 in appreciable yield (295 mg, 53%).

¹H NMR (CDCl₃) δ 7.48-7.33 (m, 5H), 5.33 (d, 1H), 5.19 (d, 1H), 5.18-5.12 (m, 1H), 2.94-2.88 (m, 1H), 2.44 (s, 3H), 1.61 (s, 3H), 1.55 (s, 3H), 1.32 (18H).

³¹P NMR δ 15.89 (¹H-decoupled, external reference; 85% H₃PO₄, δ 0.0 ppm).

### EXAMPLE 6

The structure can be represented by formula 6:

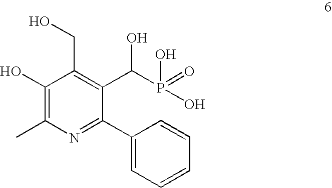

### Synthesis of [Hydroxy-(5-hydroxy-4-hydroxymethyl-6-methyl-2-phenyl-pyridin-3-yl)-methyll-phosphonic acid (6)

The fully protected phosphonate 5 (155 mg, 0.32 mmol) was hydrolyzed by heating at 60° C. in 80% aqueous acetic acid (10 mL). Co-distillation with toluene left a crude residue, which was then dissolved in methanol and precipitated with ethyl acetate to give the pure product 6 (60 mg, 57%).

¹H NMR (CDCl₃) δ 7.59-7.55 (m, 5H), 5.44-5.21 (m, 2H), 4.39 (d, 1H), 2.49 (s, 3H).

³¹P NMR δ 15.5 (¹H-decoupled, external reference; 85% H₃PO₄, δ 0.0 ppm).

### EXAMPLE 7

The structure can be represented by formula 7:

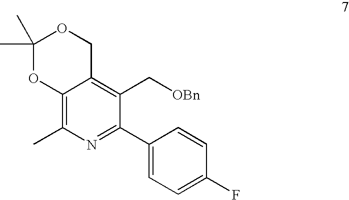

### Synthesis of 5Benzyloxymethyl-6-(4-fluoro-phenyl)-2,2,8-trimethyl4H-[1,3]dioxino[4,5-c]pyridine (7)

To a solution of N-oxide 1 (674 mg, 2.14 mmol) in tetrahydrofuran (20 mL), was added isobutylchloroformate (0.3 mL, 2.35 mmol), and the reaction mixture was stirred at room temperature under nitrogen for 10 min. The mixture was cooled to ×78° C., and then a solution of 4-fluorophenyl magnesium bromide in tetrahydrofuran (1.0 M, 7 mL, 7.0 mmol) was added. The reaction was stirred for 1 h at −78° C., then allowed to reach room temperature and further stirred for 2 h. The crude mixture was diluted with diethyl ether (100 mL) and washed exhaustively with saturated sodium bicarbonate. The organic layer was dried over anhydrous magnesium sulfate and evaporated to dryness to give the crude residue. Purification by column chromatography over silica gel gave the product 7 (229 mg) in 27% yield.

¹H NMR (CDCl₃) δ 7.47-7.45 (m, 2H), 7.33-7.30 (m, 5H), 7.06 (t,2H), 4.94 (s, 2H), 4.46 (s, 2H), 4.30 (s, 2H), 2.45 (s, 3H), 1.58 (s, 6H).

¹⁹F NMR δ −115.3 (¹H-decoupled, hexafluorobenzene δ −162.9 ppm as external standard).

### EXAMPLE 8

The structure can be represented by formula 8:

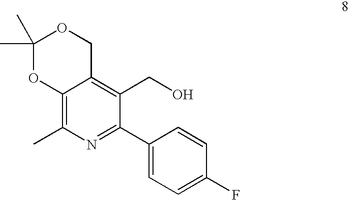

### Synthesis of [6-(4-Fluoro-phenyl)-2,2,8-trimethyl-4-[1,3]dioxino[4,5]pyridin-5yl]-methanol (8)

To a solution of compound 7 (92 mg, 0.23 mmol) in methanol (10 mL) was added 10% Pd/C (141 mg, 50% moisture content), and the reaction mixture was hydrogenated at 40 psi for 48 h. Removal of the catalyst by filtration through Celite® gave the desired compound 8 (66 mg, 95%).

¹H NMR (CDCl₃) δ 7.47-7.42 (m, 2H), 7.12-7.06 (m, 2H), 5.02 (s,2H), 4.48 (s, 2H), 2.44 (s, 3H), 1.59 (s, 6H).

### EXAMPLE 9

The structure can be represented by formula 9:

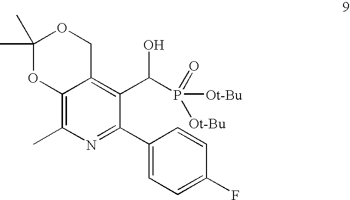

### Synthesis of {[6-(4-Fluoro-phenyl)-2,2,8-trimethyl-4H-[1,3]dioxino[4,5-cl]pyridin-5-yl]-hydroxy-methyl}-phosphonic acid di-tert-butyl ester (9)

The alcohol 8 (66 mg, 0.23 mmol) was dissolved in toluene (10 mL). To the solution was added manganese dioxide (235 mg, 2.3 mmol), and the reaction mixture was heated at 80° C. for 6 h and then at 60° C. overnight. Filtration of the reaction mixture through Celite® gave a clear solution that was evaporated to dryness to give the crude aldehyde (54 mg). The crude aldehyde was then treated with di-tert-butyl phosphite (14 mg, 0.54 mmol) using the procedure outlined in the synthesis of 5 to give desired phosphonate 9.

¹H NMR (CDCl₃) δ 7.47-7.42 (m, 2H), 7.11-7.06 (m, 2H), 5.19 (d,1H), 5.36-5.04 (m, 3H), 2.99-2.93 (m, 1H), 2.42 (d, 3H), 1.60-1.55 (m, 6H), 1.34 (d,18H).

³¹P NMR δ 14.3 (¹H-decoupled, external reference; 85% H₃PO₄, δ 0.0 ppm).

### EXAMPLE 10

The structure can be represented by formula 10:

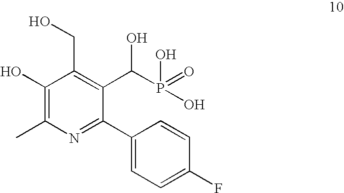

### Synthesis of {[2-(4-Fluoro-phenyl)-5-hydroxy-4hydroxymethyl-6-methyl-pyridin-3-yl]-hydroxy-methyl}-phosphonic acid (10)

The fully protected phosphonate 9 (55 mg, 0.11 mmol) was stirred in acetic acid:water 4:1 (10 mL) at 80° C. under nitrogen for 72 h. Acetic acid was removed under reduced pressure, and the residue was washed with ethyl acetate to remove non-polar impurities. The crude product was then purified by crystallization from H₂O:MeOH:diethyl ether to give 10 (23 mg, 61%).

¹H NMR (D₂O) δ 7.57-7.52 (m, 2H), 7.30-7.24 (m, 2H), 5.41-5.18 (m, 2H), 4.34 (d, 1H), 2.45 (s, 3H).

³¹P NMR δ 15.7 (¹H-decoupled, external reference; 85% H₃PO₄, δ 0.0 ppm).

### EXAMPLE 11

The structure can be represented by formula 11:

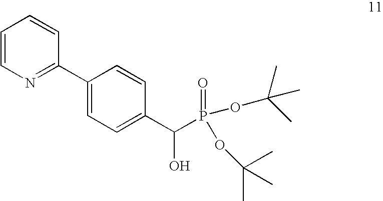

### Synthesis of [Hydroxy-(4-pyridin-2-yl-phenyl)-methyl]-phosphonic acid di-tert butyl ester (11)

To a 100 mL Schlenk-flask was added NaH (60% in mineral oil, 144 mg, 3.6 mmol) and a solution of di-tert-butyl phosphite (874 mg, 4.5 mmol) in 5 mL of THF. The mixture was stirred at room temperature under N₂ for 1 h before a solution of 4-(2-pyridyl)benzaldehyde (550 mg, 3.0 mmol) in 7 mL of THF was added. After stirring at room temperature for 2 h, the reaction was quenched with saturated aqueous NaHCO₃ and extracted with EtOAc. The organic layers were combined, dried over MgSO₄ and concentrated to dryness. The resulting powder was washed with hexane to give 751 mg (66%) of 11 as a white solid.

¹H NMR (CDCl₃) δ 8.70-8.67 (m, 1H), 7.97 (d, 2H), 7.74-7.72 (m, 2H), 7.56 (d, 2H), 7.23-7.19 (m, 1H), 4.91 (d, IH), 1.43 (m, 18H).

³¹P NMR δ 14.0 (H-decoupled, external reference; 85% H₃PO₄, δ 0.0 ppm).

### EXAMPLE 12

The structure can be represented by formula 12:

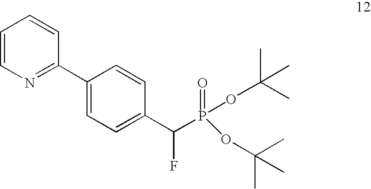

### Synthesis of [Fluoro-(4-pyridin-2-yl-phenyl)-methyl]-phosphonic acid di-tert butyl ester (12)

The hydroxyphosphonate 11 (340 mg, 0.9 mmol) was dissolved in dry dichloromethane (15 mL), and the solution was cooled. to −78° C. under nitrogen. DAST ((diethylamino)sulfur trifluoride) (0.18 mL, 218 mg, 1.35 mmol) was added. The reaction mixture was stirred at −78° C. for 0.5 h, and then at room temperature for 0.5 h before quenching with saturated aqueous NaHCO₃. The crude product was then extracted with dichloromethane (2×20 mL), dried over MgSO₄, and concentrated to dryness. The resulting crude product was purified by flash chromatography over silica gel using hexane:ethyl acetate 1:1 as eluent to give 241 mg (70%) of a-fluorophosphonate 12.

¹H NMR (CDCl₃) δ 8.71-8.68 (m, 1H), 8.02 (d, 2H), 7.76-7.74 (m,2H), 7.57-7.54 (m, 2H), 7.25-7.21 (m, 1H), 5.55 (dd, 1H), 1.45 (d, 18H).

³¹P NMR δ 7.15 (d) (¹H-decoupled, external reference; 85% H₃PO₄, δ 0.0 ppm).

¹⁹F NMR δ 200.1 (d) (¹H-decoupled, hexafluorobenzene δ −162.9 ppm as external standard).

### EXAMPLE 13

The structure can be represented by formula 13.

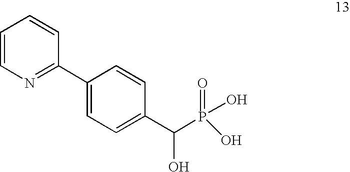

### Synthesis of [Hydroxy-(4-pyridin-2-yl-phenyl)-methyl]-phosphonic acid (13)

The fully protected phosphonate 11 (266 mg, 0.70 mmol) was stirred in 15 mL aqueous acetic acid at 80° C. under nitrogen for 72 h. Acetic acid was evaporated under reduced pressure, and the residue washed with ethyl acetate to remove non-polar impurities. The crude product was then purified by crystallization from water to give 13 (179 mg, 92%) as a white solid.

¹H NMR (0.01 N NaOH in D₂O) δ 8.57 (d, 1H), 7.97-7.84 (m, 4H), 7.62-7.59 (m, 2H), 7.43-7.36 (m, 1H), 4.86-4.82 (m, 1H).

³¹P NMR δ 16.28 (¹H-decoupled, external reference; 85% H₃PO₄, δ 0.0 ppm).

### EXAMPLE 14

The structure can be represented by formula 14

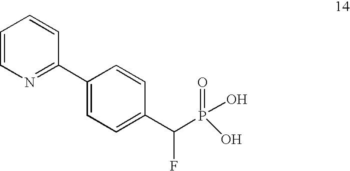

### Synthesis of [Fluoro-(4-pyridin-2-yl-phenyl)-methyl]-phosphonic acid (14)

The fully protected phosphonate 12 (188 mg, 0.5 mmol) was stirred in 10 mL aqueous acetic acid at 80° C. under nitrogen for 22 h. Acetic acid was evaporated under reduced pressure, and the residue washed with ethyl acetate to remove non-polar impurities. The crude product was then purified by crystallization from water to furnish 14 (108 mg, 81%) as a white solid.

¹H NMR (0.01 N NaOH in D₂O) δ 8.57-8.55 (m, 1H), 7.93-7.82 (m, 4H), 7.61 (d, 2H), 7.43-7.38 (m,1H), 5.62 (dd,1H).

³¹P NMR δ 11.42 (d) (¹H-decoupled, external reference; 85% H₃PO₄, δ 0.0 ppm).

¹⁹F NMR δ −195.28 (d) (¹H-decoupled, hexafluorobenzene δ −162.9 ppm as external standard).

### EXAMPLE 15

The structure can be represented by formula 15.

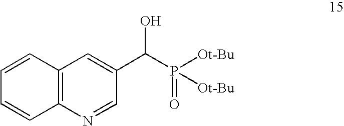

### Synthesis of (Hydroxy-quinolin-3-yl-methyl)-phosphonic acid di-tert-butyl ester (15)

Di-tert-butyl phosphite (957 mg, 4.9 mmol) was added to a suspension of NaH (60% in mineral oil, 193 mg, 4.83 mmol) in THF (5 mL) at 0° C. under nitrogen atmosphere. The solution was warmed to room temperature and stirred for 15 min, then cooled to 0° C. To this solution was added quinoline-3-carboxaldehyde (423 mg, 2.75 mmol) in THF (2 mL). The resulting solution was slowly warmed to room temperature, and stirred for 45 min. The reaction was poured into water (10 mL), extracted with diethyl ether (4×), and the organic phases were back washed with water (3×) then brine (2×). The combined organic phases were dried (MgSO₄), filtered, and evaporated to obtain the crude product as colorless oil. The crude product crystallized when kept at 5° C. overnight. The crystals were filtered and washed with hexane, then diethyl ether (quickly) to give 617 mg of crystalline 15. The volume of mother liquor was reduced to precipitate a second crop of crystals that were quickly washed with diethyl ether, then hexane to give an additional 90 mg of 15 as colorless powder. The remaining mother liquor was purified by chromatography over silica gel on a silica gel column to give 153 mg of 15 for a total combined yield of 860 mg (89%).

¹H-NMR (CDCl₃) δ 8.95 (s, 1H), 8.28 (s, 1H), 8.08 (d, 1H), 7.80 (d, 1H), 7.72-7.61 (m, 1H), 7.58-7.47 (m, 1H), 5.09 (d, 1H), 1.47 (s, 9H), 1.41 (s, 9H).

³¹P-NMR (CDCl₃) δ 13.15 (s) (¹H-decoupled, external reference; 85% H₃PO₄, δ 0.0 ppm).

### EXAMPLE 16

The structure can be represented by formula 16:

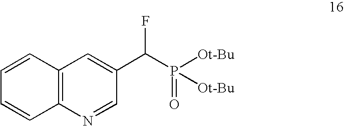

### Synthesis of (Fluoro-quinolin-3-yl-methyl)-phosphonic acid di-tert-butyl ester (16)

DAST (245 mg, 154 mmol) was added to a solution of 15 (236 mg, 0.67 mmol) in dichloromethane (5 mL) at −78° C. under nitrogen atmosphere. The resulting solution was slowly warmed to rt (˜2 h), and then stirred for another 1 h. Saturated aqueous NaHCO₃ (10 mL) was added to quench the reaction. The product was extracted with dichloromethane (4×), and the organic phase was dried (MgSO4), filtered and evaporated. The crude mixture was purified on a silica gel column using hexane to hexane:ethyl acetate 3:7 as eluent to give 16 as a yellow solid (137 mg, 58%).

¹H-NMR (CDCl₃) δ 8.95 (s, 1H), 8.28 (s, 1H), 8.12 (d, 1H), 7.74 (d, 1H), 7.63-7.52 (m, 1H), 5.70 (d, 1H), 1.47 (s, 9H), 1.46 (s, 9H).

³¹P-NMR (CDCl₃) δ 6.26 (d) (¹H-decoupled, external reference; 85% H₃PO₄, δ 0.0 ppm).

¹⁹F-NMR (CDCl₃) δ −201.7 (d) (¹H-decoupled, hexafluorobenzene δ −162.9 ppm as external standard).

### EXAMPLE 17

The structure can be represented by formula 17:

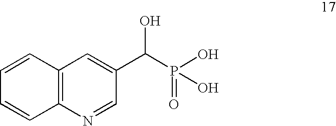

### Synthesis of (Hydroxy-guinolin-3-yl-methyl)-phosphonic acid (17)

Protected phosphonate 15 (208 mg, 0.59 mmol) was stirred in acetic acid:water 4:1 (5 mL) at 75° C. for 18 h. The suspension was cooled to room temperature, and the product was collected by filtration, washed with methanol, then ethyl acetate to give 122 mg (86%) of 17 as colorless solid.

¹H-NMR (0.1 M NaOH in D₂O) δ 8.92 (s, 1H), 8.38 (s, 1H), 8.07-7.95 (m, 2H), 7.83-7.72 (m, 2H), 7.70-7.59 (m, 1H), 4.95 (d, 1H).

³¹P-NMR (0.1 M NaOH in D₂O) δ 15.3 (s) (¹H-decoupled, external reference; 85% H₃PO₄, δ 0.0 ppm).

### EXAMPLE 18

The structure can be represented by formula 18:

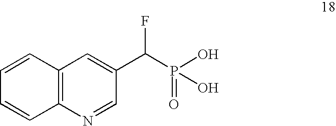

### Synthesis of (Fluoro-quinolin-3-yl-methyl)-phosphonic acid (18)

Protected phosphonate 16 (125 mg, 0.35 mmol) was stirred in acetic acid:water 4:1 (5 mL) at 75° C. for 3 h. The solvent was evaporated and the product was collected by filtration, then washed with methanol (15 mL) and dichloromethane (10 mL) to give 75 mg (89%) of 18 as colorless solid.

¹H-NMR (D₂O) δ 9.17-9.07 (m, 2H), 8.35-8.07 (m, 3H), 8.02-7.92 (m, 1H), 6.08 (m, 1H).

³¹P-NMR (D₂O) δ 9.69 (dd); ¹H-decoupled δ 9.69 (d) (external reference; 85% H₃PO₄, δ 0.0 ppm).

¹⁹F-NMR (D₂O) δ −201.8 (dd); ¹H-decoupled δ −201.8 (d) (hexafluorobenzene δ −162.9 ppm as external standard).

### EXAMPLE 19

The structure can be represented by formula 19:

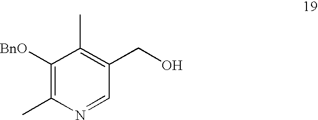

### Synthesis of (5-Benzyloxy-4,6-dimethyl-pyridin-3-yl)-methanol (19)

A mixture of 4-deoxypyridoxine hydrochloride (358 mg, 1.89 mmol), potassium carbonate (4.45 g, 32 mmol) and benzyl chloride (1.24 g, 9.7 mmol) was stirred in dry DMF (20 mL) under nitrogen atmosphere for 5 h. Water was added to quench the reaction, and the solvents were evaporated. Water (20 mL) was again added, and the organic compound was extracted with dichloromethane. The organic layer was dried (MgSO₄), filtered and evaporated to give a crude product. The crude product was purified by column chromatography over silica gel using hexane:ethyl acetate 1:1 to ethyl acetate as eluent to give 327 mg (71%) of pure 19 as an off-white solid.

¹H NMR (CDCl₃) δ 8.09 (s, 1H), 7.55-7.30 (m, 5H), 4.80 (s, 2H), 4.65 (s, 2H), 2.48 (s, 3H,), 2.31 (s, 3H).

¹³C-NMR (CDCl₃) δ 152.0, 151.8, 143.4, 139.4, 136.7, 134.3, 128.6, 128.3, 127.9, 74.7, 60.6, 19.3, 11.5.

### EXAMPLE 20

The structure can be represented by formula 20:

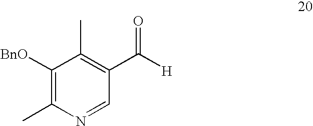

### Synthesis of 5-Benzyloxy-4,6-dimethyl-pyridine-3-carbaldehyde (20)

Alcohol 19 (235 mg, 0.96 mmol) and MnO₂ (787 mg, 7.96 mmol) were dissolved in toluene (20 mL), and the reaction mixture heated at 50° C. for 2 h. The MnO₂ was filtered off with the aid of Celite®, and the mother liquor was evaporated to give 226 mg (quantitative) of the corresponding aldehyde 20 as a light yellow solid.

¹H NMR (CDCl₃) δ 10.22 (s, 1H), 8.64 (s, 1H), 7.50-7.30 (m, 5H), 4.85 (s, 2H), 2.59 (s, 6H).

MS (ES⁺) m/z: 242 (M+H⁺).

### EXAMPLE 21

The structure can be represented by formula 21:

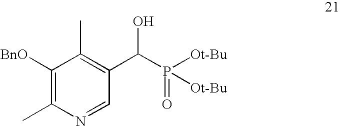

### Synthesis of [5-Benzyloxy-(4,6-dimethyl-pyridin-3-yl)-hydroxy-methyl]-phosphonic acid di-tert-butyl ester (21)

A mixture of aldehyde 20 (918 mg, 3.8 mmol), di-tert-butyl phosphite (1.03 g, 5.3 mmol) and DBU (1.5 mL, 10 mmol) was stirred in dichloromethane (30 mL) at room temperature for 24 h. Water was added to the reaction, and the mixture was extracted with dichloromethane. The organic layer was dried (MgSO₄), filtered and evaporated. The product was purified on a silica gel column using hexane:ethyl acetate 1:1 to ethyl acetate as eluent to obtain 1.37 g (83%) of 21 as a colorless solid.

¹H NMR (CDCl₃) δ 8.44 (d, 1H), 7.50-7.30 (m, 5H), 5.01 (d, 1H), 4.97 (d, 1H), 4.75 (d, 1H), 2.50 (d, 3H), 2.34 (d, 3H), 1.48 (s, 9H), 1.42 (s, 9H).

¹³C NMR (CDCl₃) δ 151.4, 151.1, 144, 138.4, 136.8, 131.4, 128.6, 128.3, 127.9, 84.1, 83.9, 74.8, 68.0, 30.5, 30.4, 19.5, 12.1.

³¹P NMR (CDCl₃) δ 13.8 (¹H-decoupled, external reference; 85% H₃PO₄, δ 0.0 ppm).

### EXAMPLE 22

The structure can be represented by formula 22:

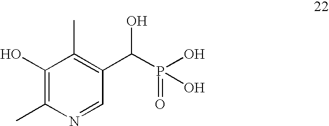

### Synthesis of [Hydroxy-(5-hydroxy-4,6-dimethyl-pyridin-3-yl)-methyl]-phosphonic acid (22)

Compound 21 (37 mg, 0.085 mmol) was hydrogenated at 30 psi in the presence of a catalytic amount of 10% Pd/C (50% moisture content) in methanol (5 mL) for 2 h. The catalyst was filtered off with the aid of Celite® to give the corresponding debenzylated compound. This compound was heated in acetic acid:water 4:1 (5 mL) at 70° C. for 3 h. The solvent was evaporated, and the product was precipitated in water. The solid was collect by filtration, and washed with methanol, then ethyl acetate to give 13 mg (64%) of 22 as a colorless solid.

¹H NMR (D₂O) δ 8.21 (s, 1H), 5.22 (d, 1H), 2.61 (d, 3H), 2.47 (s, 3H).

¹H NMR (0.1 M NaOH in D₂O) δ 7.75 (d, 1H), 4.93 (d, 1H), 2.36 (d, 3H), 2.24 (s, 3H).

³¹P NMR (0.1 M NaOH in D₂O) δ 15.7 (d) (¹H-decoupled, external reference; 85% H₃PO₄, δ 0.0 ppm).

### EXAMPLE 23

The structure can be represented by formula 23:

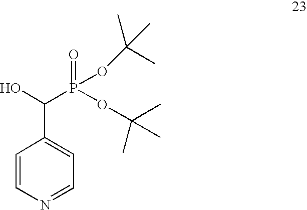

### Synthesis of (Hydroxy-pyridin-4-yl-methyl)-phosphonic acid di-tert-butyl ester (23)

To a suspension of NaH (1.6 g, 40 mmol, 60% in mineral oil) in dry THF (10 mL) was added a solution of di-tert-butyl phosphite (9.7 g, 50 mmol) in THF (10 mL). After stirring at room temperature under nitrogen for 2 h, a solution of 4-pyridinecarboxaldehyde (1.07 g, 10 mmol) in THF (30 mL) was added, and the reaction mixture was stirred at room temperature for 4 h. The reaction mixture was then quenched with saturated aqueous NaHCO₃ and extracted with ether. The organic layer was dried (MgSO₄) and concentrated to dryness. The crude product was purified by the crystallization from ether:hexane and the crystals collected by filtration to furnish 1.1 g (37%) of pure 23. This compound was used directly in the next step without further purification.

¹H NMR (CDCl₃) δ 8.52 (d, 2H), 7.41-7.39 (m, 2H), 4.86 (d, 1H), 1.42 (18H).

### EXAMPLE 24

The structure can be represented by formula 24:

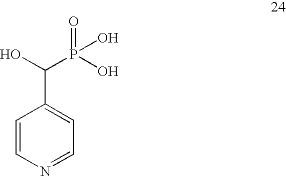

### Synthesis of (Hydroxy-pyridin-4-yl-methyl)-phosphonic acid (24)

Di-tert-butyl ester 23 (334 mg, 1.11 mmol) was dissolved in HOAc:H₂O 4:1 and stirred at 80° C. overnight. The solvents were evaporated to give a white solid that was then washed with methanol and dried under vacuum to give 160 mg (76%) of 24.

¹H NMR (D₂O) δ 8.66 (d, 2H), 8.03 (d, 2H), 5.16 (d, 1H).

³¹P NMR (D₂O) δ 13.5 (¹H-decoupled, external reference; 85% H₃PO₄, δ 0.0 ppm).

### EXAMPLE 25

The structure can be represented by formula 25:

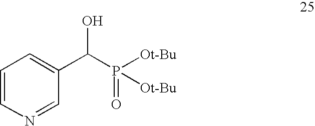

### Synthesis of (Hydroxy-pyridin-3-yl-methyl)-phosphonic acid di-tert-butyl ester (25)

Di-tert-butyl phosphite (637 mg, 3.28 mmol) was added to a solution of NaH (60% in mineral oil, 96 mg, 2.4 mmol) in THF (7 mL) at 0° C. under nitrogen atmosphere. The solution was allowed to slowly warm to room temperature, and 3-pyridinecarboxaldehyde (191 mg, 1.78 mmol) added. The resulting solution was stirred for 2 h, and then quenched with saturated aqueous sodium bicarbonate (20 mL). The aqueous solution was extracted with diethyl ether and the organic layer was dried with MgSO4, filtered and evaporated. The resulting oil was purified by column chromatography over silica gel using gradient elution (hexane to ethyl acetate to ethyl acetate:methanol 19:1) to obtain pure 25 as a colorless solid (157 mg, 29%).

¹H-NMR (CDCl₃) δ 8.62 (br s, 1H), 8.53-8.45 (m, 1H), 7.88-7.79 (m, 1H), 7.29-7.20 (m, 1H), 4.87 (d, 1H), 1.44 (s, 9H), 1.40 (s, 9H).

³¹P-NMR (CDCl₃) δ 13.2 (d) (¹H-decoupled, external reference; 85% H₃PO₄, δ 0.0 ppm).

### EXAMPLE 26

The structure can be represented by formula 26:

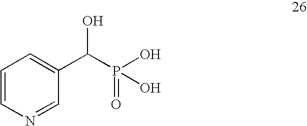

### Synthesis of (Hydroxy-pyridin-3-yl-methyl)-phosphonic acid (26)

Compound 25 (106 mg, 0.35 mmol) was heated in acetic acid:water 4:1 at 70° C. for 17 h, after which time the solvent was evaporated and the colorless solid washed with methanol to give 53 mg (79%) of 26 as a colorless solid.

¹H-NMR (D₂O) δ 8.77 (br s, 1H), 8.74-8.64 (m, 1H), 8.63-8.55 (m, 1H), 8.07-7.97 (m, 1H), 5.11 (d, 1H).

³¹P-NMR (D₂O) δ 14.7 (¹H-decoupled, external reference; 85% H₃PO₄, δ 0.0 ppm).

### EXAMPLE 27

The structure can be represented by formula 27:

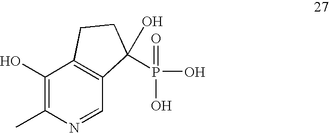

### Synthesis of (3,7-Dihydroxy-6-methyl-1,3-dihydro-furo[3,4-c]pyridin-3-yl)-phosphonic acid (27)

To a solution of hydroxy-(2,2,8-trimethyl-4H-[1,3]dioxino[4,5-c]pyrid-in-5-yl)-methyl]-phosphonic acid di-tert-butyl ester¹ (3.5 g, 8.7 mmol) in dichloromethane (100 mL) at −8° C., under nitrogen atmosphere, was added diisopropylethylamine (3.39 g, 26.2 mmol) followed by a solution of SO₃-pyridine complex (4.17 g, 26.2 mmol) in DMSO (20 mL). The yellow solution was stirred for 1.5 h at this temperature and then diluted with diethyl ether (300 mL). The organic solution was successively washed with water, saturated aqueous NaHCO₃ and brine, then dried (MgSO₄), filtered and concentrated to give the crude blocked α-ketophosphonate as a yellow oil. This crude material was deprotected by stirring in HOAc:H₂O 4:1 (60 mL) at 75° C. for 18 h. After this time, the solvent was evaporated and the resulting solid was washed with water, methanol and finally dichloromethane to furnish 1.85 g (86%) of 27 as an off white solid. ¹Reference: Structure V, U.S. Ser. No. 09/795,689, Haque, W

¹H-NMR (0.1M NaOH in D₂O) δ 7.73 (s, 1H), 5.25 (dd, 1H), 5.08 (d, 1H), 2.42 (s, 3H).

¹³C NMR (0.1M NaOH in D₂O) δ 158.1, 144.2, 143.1 (d), 137.8 (d), 120.5, 107.6 (d), 71.4 (d), 14.9.

³¹P-NMR (0.1 M NaOH in D₂O) δ 11.1 (s) (¹H-decoupled, external reference; 85% H₃PO₄, δ 0.0 ppm).

MS (ES⁺) m/z: 248.13 (M+H⁺).

### EXAMPLE 28

The structure can be represented by formula 28:

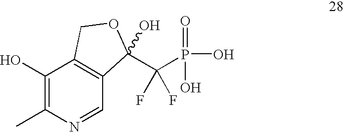

### Synthesis of [(3,7-Dihydroxy-6-methyl-1,3-dihydro-furo[3,4-c]pyridin-3-yl)-difluoromethyl]-phosphonic acid (28)

To a solution of the cyclic form of 1-difluoro-2-(5-hydroxy-4-hydroxymethyl-6-meth-yl-pyridin-3-yl)-2-oxo-ethyl]-phosphonic acid diethyl ester¹ (1.05 g, 2.97 mmol) in dry acetonitrile (20 mL) was added excess trimethylsilyl bromide (3.13 mL, 17.8 mmol), and the reaction was stirred at r.t overnight. The solvent was then evaporated, ammonium hydroxide added and the mixture stirred at room temperature for 10 min. The solution was again evaporated to dryness. Column chromatography over reverse phase (C-18) silica using CH₃CN:H₂O 9:1 as eluent gave 590 mg (60%) of 28 as a white solid. ¹References: Structure XXVII, U.S. Ser. No. 09/795,689, Haque, W.

¹H NMR (D₂O) δ 7.91 (s, 1H), 5.31 (d, 1H), 5.21 (d, 1H), 2.53 (s, 3H).

¹³C NMR (D₂O) δ 157.19, 145.0, 143.53, 133.93, 121.8, 71.0, 14.9.

¹⁹F NMR (D₂O) δ −121.80 (dd), −119.52 (dd) (¹H-decoupled, hexafluorobenzene δ −162.9 ppm as external standard).

³¹P NMR (D₂O) d 4.40 (br t) (¹H-decoupled, external reference; 85% H₃PO₄, δ 0.0 ppm).

MS (ES⁺) m/z: 298.1 (M+H⁺), (ES⁻) m/z: 296.2 (M−H⁺).

### EXAMPLE 29

The structure can be represented by formula 29:

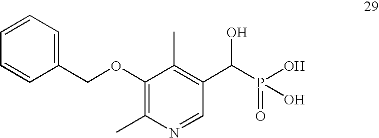

### Synthesis of [(5-Benzyloxy-4,6-dimethyl-pyridin-3-yl)-hydroxy-methyl]-phosphonic acid (29)

Compound 21 (101 mg, 0.23 mmol) was heated in acetic acid:water 4:1 at 70° C. for 2 h. The solvent was evaporated, and the resulting solid was washed with methanol, and then dichloromethane to give the colorless solid 29 (60 mg, 68%) as the acetic acid salt.

¹H NMR (DMSO-d₆) δ 8.33 (d, 1H), 7.55-7.32 (m, 5H), 4.86 (d, 1H), 4.78 (s), 2.52 (d, 3H), 2.34 (s, 3H), 1.92 (s, 3H).

³¹P-NMR (CDCl₃) δ 19.1 (¹H-decoupled, external reference; 85% H₃PO₄, δ 0.0 ppm).

### EXAMPLE 30

The structure can be represented by formula 30:

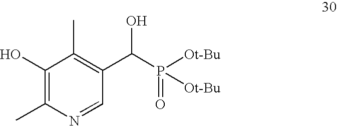

### Synthesis of [Hydroxy-(5-hydroxy-4,6-dimethyl-pyridin-3-yl)-methyl]-phosphonic acid di-tert-butyl ester (30)

A solution of compound 21 (101 mg, 0.23 mmol) in methanol containing a catalytic amount of 10% Pd/C (50% water content) was hydrogenated (30 psi) overnight. The mixture was filtered through Celite® and the solvent was evaporated to obtain 30 as a colorless solid (60 mg, 68%).

¹H NMR (CD₃OD) δ 8.10 (d, 1H), 5.04 (d, 1H, d), 4.84 (s, 2H), 2.43 (d, 3H), 2.34 (d, 3H), 1.48 (s, 9H), 1.47 (s, 9H).

³¹P-NMR (CD₃OD) δ 14.29 (¹H-decoupled, external reference; 85% H₃PO₄, δ 0.0 ppm).

### EXAMPLE 31

The structure can be represented by formula 31:

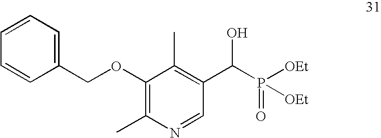

### Synthesis of [(5-Benzyloxy-4,6-dimethyl-pyridin-3-yl)-hydroxy-methyl]-phosphonic acid diethyl ester (31)

A mixture of diethyl phosphite (560 mg, 4.05 mmol), 3-O-benzyl-4-deoxypyridoxal 20 (534 mg, 2.21 mmol) and DBU (0.6 mL, 4.0 mmol) in dichloromethane (20 mL) was stirred at rt for 2 h. The solvent was evaporated and the crude oil was purified on a column of silica gel using ethyl acetate:hexane 1:1 to 1:0 for gradient elution to give 31 (593 mg, 71%) as an off white solid.

¹H-NMR (CDCl₃) δ 8.41 (s, 1H), 7.52-7.31 (m, 5H), 5.21 (d, 1H), 4.82 (s, 2H), 4.25-4.00 (m, 4H), 2.53 (d, 3H), 2.30 (d, 3H), 1.30 (t, 3H), 1.29 (t, 3H).

³¹P-NMR (CDCl₃) δ 21.6 (¹H-decoupled, external reference; 85% H₃PO₄, δ 0.0 ppm).

### EXAMPLE 32

The structure can be represented by formula 32:

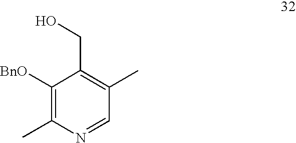

### Synthesis of (3-Benzyloxy-2,5-dimethyl-pyridin-4-yl)-methanol (32)

Benzyl chloride (6 mL, 52.1 mmol) was added to a suspension of 5-deoxypyridoxine¹ (1.63 g, 10.6 mmol) and potassium carbonate (15.2 g, 110 mmol) in dry DMF (100 mL) at rt under nitrogen atmosphere. The resulting mixture was stirred for 5 h, after which time water (10 mL) was added and the solvents were evaporated. Water (100 mL) was again added and the aqueous mixture was extracted with dichloromethane (3×). The organic phase was dried (MgSO4), filtered, and evaporated, and the crude product was purified,-by silica gel chromatography using hexane:ethyl acetate 1:1 to furnish 32 (1.60 g, 62%) as a colorless solid. ¹Reference: Muhlradt and Snell, J. Med. Chem. 10, 129-130 (1967).

¹H-NMR (CDCl₃) δ 8.13 (s, 1H), 7.50-7.30 (m, 5H), 4.92 (s, 2H), 4.64 (d, 2H), 2.54 (s, 3H), 2.33 (s, 3H), 1.96 (d, 1H).

### EXAMPLE 33

The structure can be represented by formula 33:

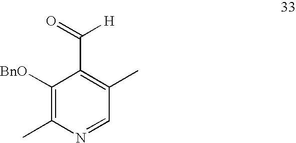

### Synthesis of 3-Benzyloxy-2,5-dimethyl-pyridine-4-carbaldehyde (33)

Alcohol 32 (906 mg, 3.73 mmol) and MnO₂ (85%; 1.64 g, 16.3 mmol) were stirred in toluene (200 mL) at 55° C. for 26 h. The mixture was the filtered through Celite® and evaporated. The crude product was purified by column chromatography over silica gel using hexane:ethyl acetate 17:3 as eluent to give aldehyde 33 (632 mg, 70%) as a colorless solid.

¹H-NMR (CDCl₃) δ 10.44 (s, 1H), 8.25 (s, 1H), 7.50-7.22 (m, 5H), 4.95 (s, 2H), 2.57 (s, 3H), 2.47 (s, 3H).

### EXAMPLE 34

The structure can be represented by formula 34:

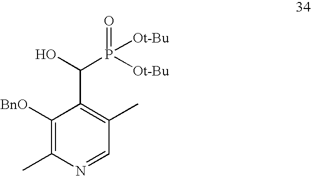

### Synthesis of [(3-Benzyloxy-2,5-dimethyl-pyridin-4-yl)-hydroxy-methyl]-phosphonic acid di-tert-butyl ester (34)

Di-tert-butyl phosphite (591 mg, 3.04 mmol) was added to a suspension of sodium hydride (60% in mineral oil, 108 mg, 2.7 mmol) in THF (10 mL) at 0° C. under nitrogen atmosphere. The mixture was warmed to rt, stirred for 15 min and then cooled to 0° C. To this solution was added a solution of aldehyde 33 (390 mg, 1.61 mmol) in THF (5 mL), and the resulting mixture was warmed to rt and stirred for 15 min. The reaction was quenched with saturated aqueous NaHCO₃ (20 mL). The organic compound was extracted with ether (3×50 mL), and the organic phase was dried (MgSO₄), filtered and evaporated. The crude product was purified by silica gel column chromatography (hexane:ethyl acetate 5:1 to 3:7). Compound 34 (667 mg, 95%) was obtained as a colorless solid.

¹H-NMR (CDCl₃) δ 8.00 (s, 1H), 7.54-7.43 (m, 2H), 7.40-7.24 (m, 3H), 5.32-5.05 (m, 2H), 4.87 (d, 1H), 4.75-4.55 (m, 1H), 2.48 (s, 3H), 2.39 (s, 3H), 1.44 (s, 9H), 1.29 (m, 9H).

³¹P-NMR (CDCl₃) δ 13.8 (¹H-decoupled, external reference; 85% H₃PO₄, δ 0.0 ppm).

### EXAMPLE 35

The structure can be represented by formula 35:

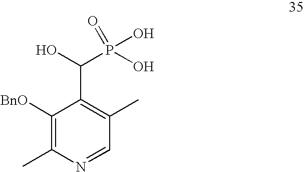

### Synthesis of [(3-Benzyloxy-2,5-dimethyl-pyridin-4-yl)-hydroxy-methyl]-phosphonic acid (35)

Protected phosphonate 34 (107 mg, 0.24 mmol) was stirred in acetic acid:water 4:1 (5 mL) for 3 h at 70° C. The solvent was evaporated, the crude product dissolved in water and the solution was kept at 5° C. overnight. The precipitated solid was collected by filtration and washed with water, methanol, then ethyl acetate to give 60 mg (75%) of 35 as a colorless solid.

¹H-NMR (D₂O) δ 8.18 (s, 1H), 7.65-7.40 (m, 5H), 5.61 (d, 1H), 5.21 (d, 1), 5.04 (d, 1H), 2.67 (s, 3H), 2.62 (s, 3H).

³¹P-NMR (D₂O) δ 14.38 (¹H-decoupled, external reference; 85% H₃PO₄, δ 0.0 ppm).

### EXAMPLE 36

The structure can be represented by formula 36:

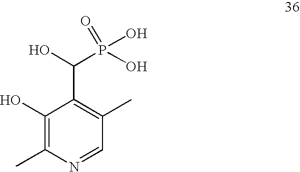

### Synthesis of [Hydroxy-(3-hydroxy-2,5-dimethyl-pyridin-4-yl)-methyl]-phosphonic acid (36)

A solution of the a-hydroxy phosphonate 34 (115 mg, 0.26 mmol) in methanol (5 mL) containing 10% Pd/C (50% moisture content) was hydrogenated (30 psi) at rt for 2 h. The palladium catalyst was removed by filtration through Celite® and the solvents were removed. The resulting product was then dissolved in acetic acid:water 4:1 (5 mL) and stirred at 70° C. for 3 h. The solvent was evaporated and the product was precipitated in ice water. The solid was filtered off and washed with ethyl acetate to yield 36 as a colorless solid (43 mg, 70% overall yield for two steps).

¹H-NMR (0.1M NaOH in D₂O) δ 7.39 (s, 1H), 4.91 (d, 1H), 2.24 (s, 3H), 2.21 (s, 3H).

¹³C-NMR (0.1M NaOH in D₂O) δ 158.6, 146.8, 134.8, 134.6, 131.1, 72.5 (d, J=145.1 Hz), 18.2, 16.3.

³¹P-NMR (0.1M NaOH in D₂O) δ 17.7 (¹H-decoupled, external reference; 85% H₃PO₄, δ 0.0 ppm).

MS (ES⁺) m/z: 234.4 (M+H⁺).

### EXAMPLE 37

The structure can be represented by formula 37:

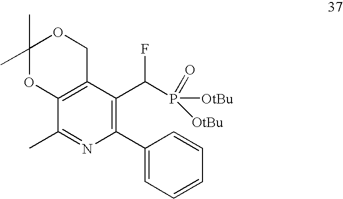

### Synthesis of [Fluoro-(2,2,8-trimethyl-6-phenyl-4H-[1,3]dioxino[4,5-c]pyridin-5-yl)-methyl]-phosphonic acid di-tert-butyl ester (37)

Compound 5 (1.25 g, 2.6 mmol) was dissolved in dry dichloromethane (35 mL) and cooled down to −78° C. under nitrogen. DAST (0.5 mL, 0.63 g, 3.9 mmol) was added and the temperature was allowed to slowly warm to −40° C. over 2.5 h then to room temperature for 0.5 h. The reaction was quenched with saturated aqueous NaHCO₃ and extracted with dichloromethane. The organic solution was dried (MgSO₄), filtered and evaporated to dryness. The resulting crude product was purified with by flash chromatography over silica gel (hexane:ethyl acetate 4:1) to give 680 mg (54%) of 37 as a colorless solid.

¹H NMR (CDCl₃) δ 7.52-7.49 (m, 2H), 7.44-7.34 (m, 3H), 5.84 (dd, 1H), 5.66 (m, 1H), 5.02 (m, 1H), 2.46 (s, 3H), 1.61 (s, 3H), 1.56 (s, 3H), 1.47 (s, 9H), 1.29 (s, 9H).

³¹p NMR (CDCl₃) δ 8.16 (d, J_{P,F}=90.6 Hz) (¹H-decoupled, external reference; 85% H₃PO₄, δ 0.0 ppm).

¹⁹F NMR (CDCl₃) δ −200.7 (d, J_{P,F}=90.4 Hz) (¹H-decoupled, hexafluorobenzene δ −162.9 ppm as external standard).

### EXAMPLE 38

The structure can be represented by formula 38:

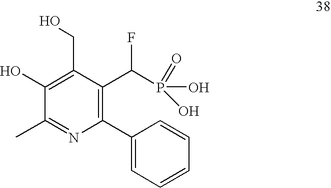

### Synthesis of [Fluoro-(5-hydroxy-4-hydroxymethyl-6-methyl-2-phenyl-pyridin-3-yl)-methyl]-phosphonic acid (38)

Compound 37 (0.68 g, 1.4 mmol) was dissolved in HOAc:H₂O 4:1 (20 mL) and stirred at 80° C. for 48 h. The solvents were removed, the residue dissolved in methanol and the crude product precipitated by the addition of diethyl ether. The resulting solid was dissolved in water and the solution was concentrated to give 80 mg of 38 as a white solid after filtration. The filtrate was evaporated and the crude material purified by flash column chromatography over silica gel (CH₂Cl₂:MeOH:NH₄OH 35:15:1) to give an additional 150 mg of 38 (combined yield 50%).

¹H NMR (CD₃OD) δ 7.56-7.45 (m, 5H), 5.38-5.32 (m, 2H), 5.08 (dd, 1H), 2.50 (s, 3H).

³¹P NMR (CD₃OD) δ 8.26 (d, J_{P,F}=68 Hz) (¹H-decoupled, external reference; 85% H₃PO₄, δ 0.0 ppm).

¹⁹F NMR (CD₃OD) δ −170 (d, J_{P,F}=68 Hz) (¹H-decoupled, hexafluorobenzene δ −162.9 ppm as external standard).

### EXAMPLE 39

Ischemia Reperfusion

Male Wistar rats (200-300 g, Tuck, Rayleigh, Essex, U.K.) were anesthetised with thiopentone sodium (Intraval®, 120 mg/kg i.p.; Rhone-Merrieux, Essex, U.K.). The rats were tracheotomised, intubated and ventilated with a Harvard ventilator (70 strokes/min, tidal volume: 8-10 mL/kg, inspiratory oxygen concentration: 30%). Body temperature was maintained at 38±1° C. The right carotid artery was cannulated and connected to a pressure transducer (MLT 1050, AD Instruments Ltd, Hastings, UK) to monitor mean arterial blood pressure (MAP) and heart rate (HR), which were continuously recorded on a data acquisition system such as Powerlab® Version 4.0.4, AD Instruments, Hastings, UK. The right jugular vein was cannulated for drug administration and the administration of Evans Blue dye (at the end of the experiment). A lateral thoracotomy was performed and the heart was suspended in a temporary pericardial cradle. A snare occluder was placed around the left anterior descending coronary artery (LAD). After completion of the surgical procedure the animals were allowed to stabilise for 30 min before LAD ligation. Five minutes prior to the onset of myocardial ischemia, a bolus injection (i.v.) of either vehicle or phosphonate was administered. The coronary artery was occluded at time 0 by tightening of the occluder. After 25 min of acute myocardial ischemia, the occluder was re-opened to allow the reperfusion for 2 h. Following the 2 h reperfusion period, the coronary artery was re-occluded and Evans Blue dye (1 mL of 2% w/v) was injected into the left ventricle/via the right jugular vein cannula, to distinguish between perfused and non-perfused (MR) sections of the heart. The Evans Blue solution stains the perfused myocardium/while the occluded vascular bed remains uncolored. The animals were killed with an overdose of anesthetic and the heart excised. It was sectioned into slices of 3-4 mm, the right ventricular wall was removed, and the MR (pink) was separated from the non-ischemic (blue) area. The MR was cut into small pieces and incubated with p-nitroblue tetrazolium (NBT, 0.5 mg/mL) for 40 min at 37° C. In the presence of intact dehydrogenase enzyme systems (viable myocardium), NBT forms a dark blue formazan, while areas of necrosis lack dehydrogenase activity and therefore fail to stain. Pieces were separated according to staining and weighed to determine the infarct size as a percentage of the weight of the AAR.

The phosphonate, for example compound 38, was suspended in 0.9% sodium chloride and the pH adjusted to pH 7 with the aid of dilute aqueous sodium hydroxide. To evaluate the effects of the phosphonates described herein on the infarct size caused by regional myocardial ischemia and reperfusion, all animals were randomised into groups (see Table 1, where N denotes the number of animals used in the study, and n represents the number of survivors). Any animals that died as a result of occlusion of the left main stem coronary artery (which results in an AAR of more than 70% of the left ventricle) are not included in Table 1.

TABLE 1
Evaluation of the Ability of Phosphonates to Reduce Infarct
Size—Experimental Design

| Group | Group I.D. | Treatment | Dose | N | n |
|---|---|---|---|---|---|
| 1 | Sham Vehicle | Saline pH 7 | 1 mL/kg i.v | 12 | 12 |
| 2 | MI Vehicle | Saline pH 7 | 1 mL/kg i.v | 11 | 11 |
| 3 | MI − 38 | Phosphonate 38 | 32 mg/kg i.v. | 10 | 10 |

The first group of animals was subjected to the surgical procedure alone not subjected to LAD occlusion) and were treated with vehicle for the test compounds. The second group comprised of animals that were subjected to regional myocardial ischemia 25 min) followed by reperfusion (2 h) and were treated with the vehicle for the test compounds. The remainder of the animals used in the study were subjected to regional myocardial ischemia (25 min) followed by reperfusion (2 h) and were treated with phosphonate (see Table 1 for details).

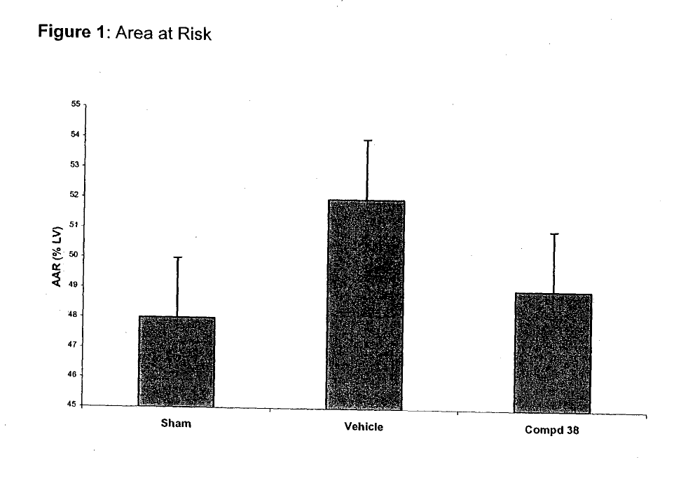

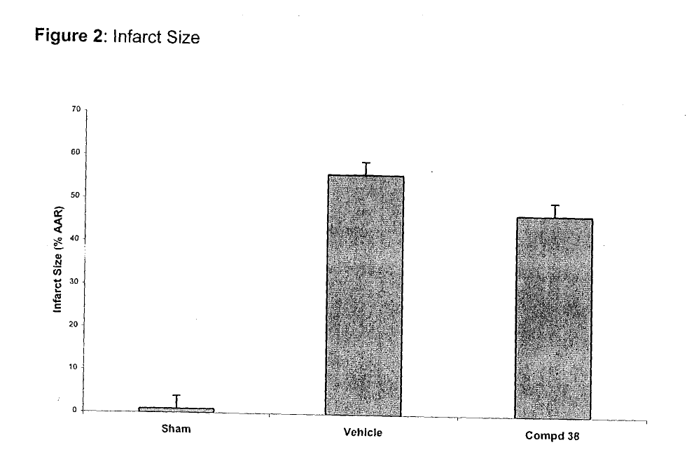

The area at risk of infarction (AAR) was similar in all three groups studied and ranged from 49±1 to 52±3 (P>0.05, FIG. 1). In rats, that were treated with vehicle, occlusion of the LAD (for 25 min) followed by reperfusion (for 2 h) resulted in an infarct size of 56±3% of the AAR (n=11). Intravenous administration of 38 (32 mg/kg i.v.) reduced myocardial infarct size (P=0.047, unpaired Students t-test, FIG. 2).

We claim:

1. A compound of general formula: 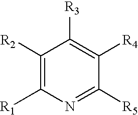
Wherein:
R₁ is selected from H and CH₃, and R₂ is selected from H and OH, or R₁ and R₂ together form an optionally substituted phenyl ring which is fused to the pyridine ring; and
R₃ is selected from H, CH₃, CH₂OH and 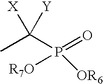
R₄ is selected from H, CH₃, CH₂OH, 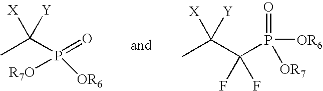
R₅ is selected from H, phenyl, halogen-substituted phenyl and 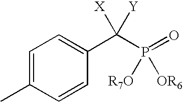
Wherein R₆ and R₇ are each independently selected from H, Na⁺, K⁺, alkyl and optionally substituted aryl, and X and Y are each independently selected from H, OH and F, or at least one of X and Y is an heteroatom and together with R₃ forms a bridge with the proviso that R₄ is 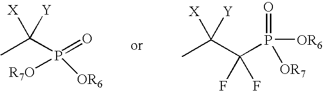
and N-oxides thereof, and biologically acceptable salts thereof.

2. The compound according to claim 1, wherein said halogen-substituted phenyl is a fluoro-substituted phenyl.

3. The compound according to claim 1, wherein said halogen-substituted phenyl is p-C₆H₄F.

4. The compound according to claim 1, wherein said heteroatom is selected from O and S.

5. The compound according to claim 1, wherein said heteroatom is O.

6. The compound according to claim 1, wherein said bridge is selected from —CH₂—, —CH₂CH₂— and —CH₂CH₂CH₂—.

7. The compound according to claim 1, wherein said bridge is a methylene bridge.

8. The compound according to claim 1, wherein said alkyl is a C₁ to C₆ straight or branched alkyl.

9. The compound according to claim 1, wherein said alkyl is t-butyl.

10. The compound according to claim 1, wherein said aryl is phenyl or naphthyl.

11. The compound according to claim 1, wherein R₁, R₂, R₄, R₅ are all H and R₃ is

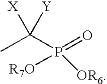

12. The compound according to claim 1, wherein R₁, R₂, R₃, R₅ are all H and R₄ is

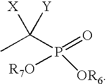

13. The compound according to claim 1, wherein R₁, R₂, R₃, R₄ are all H and R₅ is

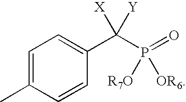

14. The compound according to claim 1, wherein R₁ and R₂ together form an optionally substituted phenyl ring which is fused to the pyridine ring; R₃ and R₅ are both H; and R₄ is

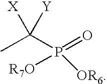

15. The compound according to claim 1, wherein R₁ and R₃ are both CH₃; R₂ is OH; R₅ is H; and R₄ is

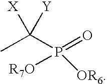

16. The compound according to claim 1, wherein R₁ and R₄ are both CH₃; R₂ is OH; R₅ is H; and R₃ is

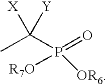

17. The compound according to claim 1, wherein R₁ is CH₃; R₂ is OH; R₃ is CH₂OH; R₅ is H; and R₄ is

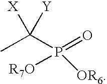

18. The compound according to claim 1, wherein R₁ is CH₃; R₂ is OH; R₃ is CH₂OH; R₅ is H; and R₄ is

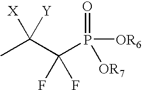

19. The compound according to claim 1, wherein R₁ is CH₃; R₂ is OH; R₃ is CH₂OH; R₅ is C₆H₅; and R₄ is

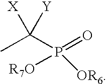

20. The compound of claim 1, wherein R₁ is CH₃; R₂ is OH; R₃ is CH₂OH; R₅ is p-C₆H₄F; and R₄ is

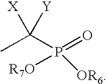

21. A compound according to claim 1, wherein R₅ is p-C₆H₄F.

22. A compound according to claim 1 selected from: [Hydroxy-(5-hydroxy-4-hydroxymethyl-6-methyl-2-phenyl-pyridin-3-yl)-methyl]-phosphonic acid; {[2-(4-Fluoro-phenyl)-5-hydroxy-4-hydroxymethyl-6-methyl-pyridin-3-yl]-hydroxymethyl}-phosphonic acid; [Hydroxy-(4-pyridin-2-yl-phenyl)-methyl]-phosphonic acid; [Fluoro-(4-pyridin-2-yl-phenyl)-methyl]-phosphonic acid; (Hydroxy-quinolin-3-yl-methyl)-phosphonic acid; (Fluoro-quinolin-3-yl-methyl)-phosphonic acid; [Hydroxy-(5-hydroxy-4,6-dimethyl-pyridin-3-yl)-methyl]-phosphonic acid; (Hydroxy-pyridin-4-yl-methyl)-phosphonic acid; (Hydroxy-pyridin-3-yl-methyl)-phosphonic acid; (3,7-Dihydroxy-6-methyl-1,3-dihydro-furo[3,4-c]pyridin-3-yl)-phosphonic acid; [(3,7-Dihydroxy-6-methyl-1,3-dihyrdo-furo[3,4-c]pyridin-3-yl)-difluoromethyl]-phosphonic acid; and nicotinyl phosphonates thereof, N-oxides thereof, phosphonate esters thereof and biologically acceptable salts thereof.

23. A compound according to claim 1 comprising:

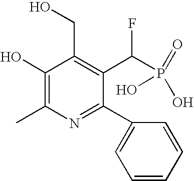

24. A pharmaceutical composition comprising a therapeutically effective amount of a compound according to claim 1 and a pharmaceutically acceptable carrier.

25. The compound according to claim 1, wherein at least one polar group is blocked by a lipophilic moiety capable of being enzymatically cleaved off after absorption into the circulatory system.

26. The compound according to claim 25, wherein said lipophilic moiety is an ester.

27. The compound according to claim 25, wherein said lipophilic moiety is a phosphonate ester.

28. A method of treating hypertension in a mammal comprising administering to the mammal a therapeutically effective amount of a compound according to claim 1 in a unit dosage form.

29. A method of treating myocardial infraction in a mammal comprising administering to the mammal a therapeutically effective amount of a compound according to claim 1 in a unit dosage form.

30. A method of treating ischemia reperfusion injury in a mammal comprising administering to the mammal a therapeutically effective amount of a compound according to claim 1 in a unit dosage form.

31. A method of treating myocardial ischemia in a mammal comprising administering to the mammal a therapeutically effective amount of a compound according to claim 1 in a unit dosage form.

32. A method of treating congestive heart failure in a mammal comprising administering to the mammal a therapeutically effective amount of a compound according to claim 1 in a unit dosage form.

33. A method of treating arrhythmia in a mammal comprising administering to the mammal a therapeutically effective amount of a compound according to claim 1 in a unit dosage form.

34. A method of reducing blood clots in a mammal comprising administering to the mammal a therapeutically effective amount of a compound according to claim 1 in a unit dosage form.

35. A method of treating hypertrophy in a mammal comprising administering to the mammal a therapeutically effective amount of a compound according to claim 1 in a unit dosage form.

36. A method of treating a disease that arises from thrombotic and prothrombotic states in which the coagulation cascade is activated in a mammal comprising administering to the mammal a therapeutically effective amount of a compound according to claim 1 in a unit dosage form.

37. A method of treating diabetes mellitus in a mammal comprising administering to the mammal a therapeutically effective amount of a compound according to (a) in a unit dosage form.

38. A method of treating insulin resistance in a mammal comprising concurrently administering to the mammal a therapeutically effective amount of a compound according to claim 1 in a unit dosage form.

39. A method of treating hyperinsulinemia in a mammal comprising administering to the mammal a therapeutically effective amount of a compound according to claim 1 in a unit dosage form.

40. A method of treating diabetes-induced hypertension in a mammal comprising administering to the mammal a therapeutically effective amount of a compound according to claim 1 in a unit dosage form.

41. A method of treating diabetes-related damage to blood vessels, eyes, kidneys, nerves, autonomic nervous system, skin, connective tissue, or immune system in a mammal comprising administering to the mammal a therapeutically effective amount of a compound according to claim 1 in a unit dosage form.

42. A method of treating obesity in a mammal comprising administering to the mammal a therapeutically effective amount of a compound according to claim 1 in a unit dosage form.

43. A compound according to claim 1 which is a nicotinic acid derivative.

44. A kit comprising the composition of claim 1 and instructions for its use in the treatment of a cardiovascular disease, a disease that arises from a thrombotic or prothombotic state in which the coagulation cascade is activated, diabetis, or related diseases.
